(19) United States
(12) Patent Application Publication
(10) Pub. No.: US 2019/0201713 A1
(43) Pub. Date: Jul. 4, 2019

(54) HELMET AND MODULAR CAP FOR LASER LIGHT HAIR GROWTH THERAPY

(71) Applicant: LaserStim, Inc., Plano, TX (US)

(72) Inventor: Kim Robin Segal, Plano, TX (US)

(73) Assignee: LaserStim, Inc., Plano, TX (US)

(21) Appl. No.: 16/123,085

(22) Filed: Sep. 6, 2018

Related U.S. Application Data
(60) Provisional application No. 62/555,040, filed on Sep. 6, 2017.

(51) Int. Cl. A61N 5/06

(52) U.S. Cl. CPC A61N 5/0617; A61N 2005/0628; A61N 2005/067; A61N 2005/0659; A61N 2005/0647

(57) ABSTRACT

A wearable apparatus for treatment of living biological tissue by optical irradiation.

### CROSS-REFERENCE TO RELATED APPLICATION

This application claims the benefit of U.S. patent application Ser. No. 62/555,040 titled “HELMET AND MODULAR CAP FOR LASER LIGHT HAIR GROWTH THERAPY” filed on Sep. 6, 2018, the contents of which is incorporated by reference in its entirety.

### BACKGROUND

### 1. Field

The present invention relates generally to the treatment of living biological tissue by optical irradiation

### 2. Related Art

This invention generally relates to human hair growth and, more particularly, to methods and devices for stimulating hair growth through stimulation of the hair follicles by means of a laser.

Alopecia (hair loss) is a major concern for the adult population. Expenditures for hair restoration products and treatments for hair loss represent a major component of the multibillion-dollar cosmetic industry in the United States. Examples of techniques for hair retention and regeneration include the use of hair weaving, the use of hairpieces, the application of hair thickening sprays and shampoos, hair transplantation, and the fashioning of coiffures which distribute hair to cover balding regions of the scalp. In addition, topical drug therapies, such as Minoxidil (Rogaine®) or oral drug therapies such as Finasteride (Propecia®), are in current use to stimulate hair growth in men suffering from male pattern baldness, i.e. baldness occurring at the crown and temples. However, this chemical cannot be used by women, can cause a negative skin reaction on the scalp, and is, therefore, not suitable for everyone, and efficacy is limited and not universal.

Diode laser systems have been developed for various medical treatments of the human body. See for example, Applicant's prior U.S. Pat. Nos. 5,755,752 and 6,033,431, which are both incorporated herein by reference in their entirety. Depending on the type of treatment desired, lasers of various wavelengths, periods of exposure and other such influencing factors have been developed.

Optical energy generated by lasers has been used for various medical and surgical purposes because laser light, as a result of its monochromatic and coherent nature, can be selectively absorbed by living tissue. The absorption of the optical energy from laser light depends upon certain characteristics of the wavelength of the light and properties of the irradiated tissue, including reflectivity, absorption coefficient, scattering coefficient, thermal conductivity, and thermal diffusion constant. The reflectivity, absorption coefficient, and scattering coefficient are dependent upon the wavelength of the optical radiation. The absorption coefficient is known to depend upon such factors as interband transition, free electron absorption, grid absorption (photon absorption), and impurity absorption, which are also dependent upon the wavelength of the optical radiation.

In living tissue, water is a predominant component and has, in the infrared portion of the electromagnetic spectrum, an absorption band determined by the vibration of water molecules. In the visible portion of the spectrum, there exists absorption due to the presence of hemoglobin. Further, the scattering coefficient in living tissue is a dominant factor.

Thus, for a given tissue type, the laser light may propagate through the tissue substantially unattenuated, or may be almost entirely absorbed. The extent to which the tissue is heated and ultimately destroyed depends on the extent to which it absorbs the optical energy. It is generally preferred that the laser light be essentially transmissive through tissues which are not to be affected, and absorbed by tissues which are to be affected. For example, when applying laser radiation to a region of tissue permeated with water or blood, it is desired that the optical energy not be absorbed by the water or blood, thereby permitting the laser energy to be directed specifically to the tissue to be treated. Another advantage of laser treatment is that the optical energy can be delivered to the treatment tissues in a precise, well-defined location such as an acupuncture point and at predetermined, limited energy levels.

Ruby and argon lasers are known to emit optical energy in the visible portion of the electromagnetic spectrum, and have been used successfully in the field of ophthalmology to reattach retinas to the underlying choroidea and to treat glaucoma by perforating anterior portions of the eye to relieve interoccular pressure. The ruby laser energy has a wavelength of 694 nanometers (nm) and is in the red portion of the visible spectrum. The argon laser emits energy at 488 nm and 515 nm and thus appears in the blue-green portion of the visible spectrum. The ruby and argon laser beams are minimally absorbed by water, but are intensely absorbed by blood chromogen hemoglobin. Thus, the ruby and argon laser energy is poorly absorbed by non-pigmented tissue such as the cornea, lens and vitreous humor of the eye, but is absorbed very well by the pigmented retina where it can then exert a thermal effect.

Another type of laser which has been adapted for surgical use is the carbon dioxide (CO2) gas laser which emits an optical beam which is absorbed very well by water. The wavelength of the CO2 laser is 10,600 nm and therefore lies in the invisible, far infrared region of the electromagnetic spectrum, and is absorbed independently of tissue color by all soft tissues having a high water content. Thus, the CO2 laser makes an excellent surgical scalpel and vaporizer. Since it is completely absorbed, its depth of penetration is shallow and can be precisely controlled with respect to the surface of the tissue being treated. The CO2 laser is thus well-suited for use in various surgical procedures in which it is necessary to vaporize or coagulate neutral tissue with minimal thermal damage to nearby tissues.

Another laser in widespread use is the neodymium doped yttrium-aluminum-garnet (Nd:YAG) laser. The Nd:YAG laser has a predominant mode of operation at a wavelength of 1064 nm in the near infrared region of the electromagnetic spectrum. The Nd:YAG optical emission is absorbed to a greater extent by blood than by water making it useful for coagulating large, bleeding vessels. The Nd:YAG laser has been transmitted through endoscopes for treatment of a variety of gastrointestinal bleeding lesions, such as esophageal varices, peptic ulcers, and arteriovenous anomalies.

The foregoing applications of laser energy are thus well suited for use as a surgical scalpel and in situations where high-energy thermal effects are desired, such as tissue vaporization, tissue cauterization, and coagulation.

Although the foregoing laser systems perform well, they commonly generate large quantities of heat and require a number of lenses and mirrors to properly direct the laser light and, accordingly, are relatively large, unwieldy, and expensive. These problems are somewhat alleviated in some systems by locating a source of laser light distal from a region of tissue to be treated and providing fiber optic cable for carrying light generated from the source to the tissue region, thereby obviating the need for a laser light source proximal to the tissue region. Such systems, however, are still relatively large and unwieldy and, furthermore, are much more expensive to manufacture than a system which does not utilize fiber optic cable. Moreover, the foregoing systems generate thermal effects, which can damage living tissue, rather then provide therapeutic treatment to the tissue.

### BRIEF DESCRIPTION OF THE DRAWINGS

The novel features believed characteristic of the invention are set forth in the appended claims. The invention itself, however, as well as a preferred mode of use, further objects and advantages thereof, will best be understood by reference to the following detailed description of an illustrative embodiment when read in conjunction with the accompanying drawings, wherein:

FIG. 1 shows a schematic diagram of a diode laser irradiation system of the present invention;

FIG. 2 shows an elevational view of a wand used in the system of FIG. 1;

FIG. 3A shows an enlarged, elevational view of a laser resonator used in the wand of FIG. 2;

FIG. 3B shows an enlarged, end view of the laser resonator used in the wand of FIG. 3A;

FIG. 4 shows a block diagram of a device for appetite suppression through stimulation of acupuncture points in the scalp, in accordance with an embodiment of the invention;

FIG. 5A shows a development view of one form of cap showing the placement of the lasers for one representative embodiment, according to embodiments of the invention;

FIG. 5B shows a development view of another form of cap illustrating the placement of the lasers for another representative embodiment, according to embodiments of the invention;

FIG. 6 shows a side view of the cap given in FIG. 5, according to an embodiment of the invention;

FIGS. 7A-7C show a modular laser system, according to an embodiment of the invention;

FIGS. 8A-8E show an interchangeable modular laser diode cap in accordance with an embodiment of the invention;

FIGS. 9A and 9B show front and side views, respectively, of a device for treating patients for hair growth stimulation, according to an embodiment of the present disclosure;

FIGS. 10A-10D show rear, side, front and bottom views, respectively, of a light application helmet according to one embodiment of the present disclosure;

FIGS. 11A and 11B show a top and side view, respectively, of the light application helmet shown in FIGS. 10A-10D, as it appears with the top cover removed; and

FIG. 12 shows an alternative embodiment in which each laser module has its own printed circuit board (PCB) control unit

### DETAILED DESCRIPTION

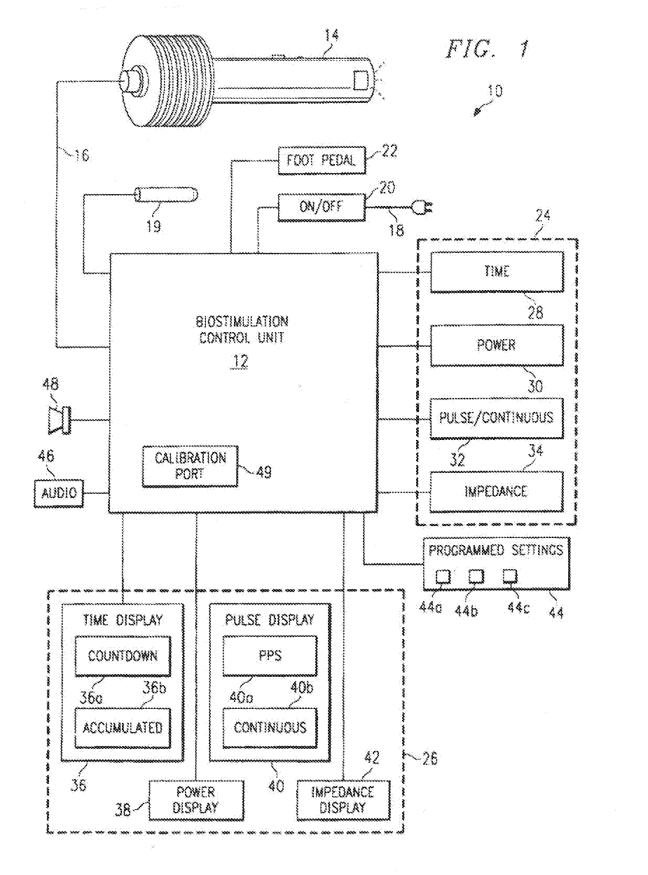

Referring to FIG. 1, the reference numeral 10 refers generally to the diode laser irradiation system of the present invention which includes a biostimulation control unit 12 for controlling the operation of a hand-operated probe, i.e., a laser treatment wand 14, electrically connected to the control unit via a coaxial cable 16. As will be described in detail below, the wand 14 houses a diode laser capable of emitting low level reactive laser light for use in tissue irradiation therapy.

The control unit 12 receives power through a power supply line 18 adapted for connection to a conventional 120-volt power outlet. A ground piece 19 is connected to the control unit 12 and is held by a patient receiving the tissue irradiation therapy to provide an electrical ground for safety purposes. An on/off switch 20 is connected in series with the line 18 for controlling the flow of power through the line. A foot pedal 22 is connected to the control unit 12 and is depressible for activating the generation and emission of laser light from the wand 14. Activation may alternatively, or additionally, be provided using a switch on the wand 14.

The control unit 12 includes laser setting controls 24 and corresponding setting displays 26. The setting controls 24 are utilized to select operational parameters of the control unit 12 to effect the rate of absorption and conversion to heat of tissue irradiated by the wand 14, according to desired treatment protocols. Generally, the treatment protocols provide for a rate of absorption and conversion to heat in the irradiated tissue in a range between a minimum rate sufficient to elevate the average temperature of the irradiated tissue to a level above the basal body temperature of the subject and a maximum rate which is less than the rate at which the irradiated tissue is converted to a collagenous substance. The treatment protocols vary time, power, and pulse/continuous mode parameters in order to achieve the desired therapeutic effects.

The setting controls 24 include a treatment time control 28, a power control 30, and a pulse/continuous mode control 32. Adjustments in treatment time, power and pulse/continuous mode operation of the wand 14 utilizing the controls 28-32 make possible improved therapeutic effects based upon the aforementioned treatment protocols involving one or more of these parameters. Also, an impedance control 34 is provided adjusting an impedance measurement of the tissue to a baseline value, according to skin resistance, as discussed further below, whereby improvements in tissue condition may be monitored. It is understood that, according to the specific embodiment of the control unit 12, the setting controls 24 may include any combination of one or more of the controls 28-34.

The setting displays 26 include a time display 36, a power display 38, a pulse display 40 and an impedance display 42. In one embodiment, each of the displays 26 are light emitting diode (LED) displays such that the corresponding setting controls 24 can be operated to increment or decrement the settings, which are then indicated on the displays. A programmed settings control 44 is used to save setting selections and then automatically recall them for convenience, using one or more buttons 44a-44c, for example.

The time control 28 adjusts the time that laser light is emitted from the wand 14, as indicated on the time display 36. The time display 36 includes a countdown display 36a and an accumulated display 36b. Once the time control 28 is set, the countdown display 36a indicates the setting so that as the wand 14 is operated the time is decremented to zero. The accumulated time display 36b increments from zero (or any other reset value) as the wand 14 is operated so that the total treatment time is displayed. The time display 36 takes into account the pulsed or continuous mode operation of the system 10.

The power control 30 adjusts the power dissipation level of the laser light from the wand 14 in a range from zero to 1000 milliwatts (mW), with typical operation ranging up to about 500 mW. The pulse/continuous mode control 32 sets the system 10 to generate laser light energy from the wand 14 either continuously or as a series of pulses. The control 32 may include, for example, a pulse duration rheostat (not shown) for adjusting the pulse-on or pulse-off time of the wand 14. In one implementation, the pulses-per-second (PPS) is set in a range from zero to 9995, adjustable in 5 step increments. The PPS setting is displayed on a PPS display 40a. The pulse duration may alternatively, or additionally, be displayed indicating the duty cycle of pulses ranging from 5 to 99 (e.g., 5 meaning that the laser is “on” 5% of the time). A continuous mode display 40b is activated when the system 10 is being operated in the continuous wattage (CW) mode of operation.

An audio volume control 46 is provided for generating an audible warning tone from a speaker 48 when laser light is being generated. Thus, for example, the tone may be pulsed when the system is operating in the pulse mode of operation.

The impedance control 34 is a sensitivity setting that is calibrated and set, according to the tissue skin resistance, to a baseline value which is then indicated on the impedance display 42. As therapy progresses the impedance readout on the display 42 changes (i.e., it decreases) thereby indicating progress of treatment.

A calibration port 49 is utilized to verify laser performance by placing the wand 14 in front of the port and operating the system 10. The port 49 determines whether the system 10 is operating within calibration specifications and automatically adjusts the system parameters.

While not shown, the control unit 12 includes digital and analog electronic circuitry for implementing the foregoing features. The details of the electronic circuitry necessary to implement these features will be readily understood by one of ordinary skill in the art in conjunction with the present disclosure and therefore will not be described in further detail.

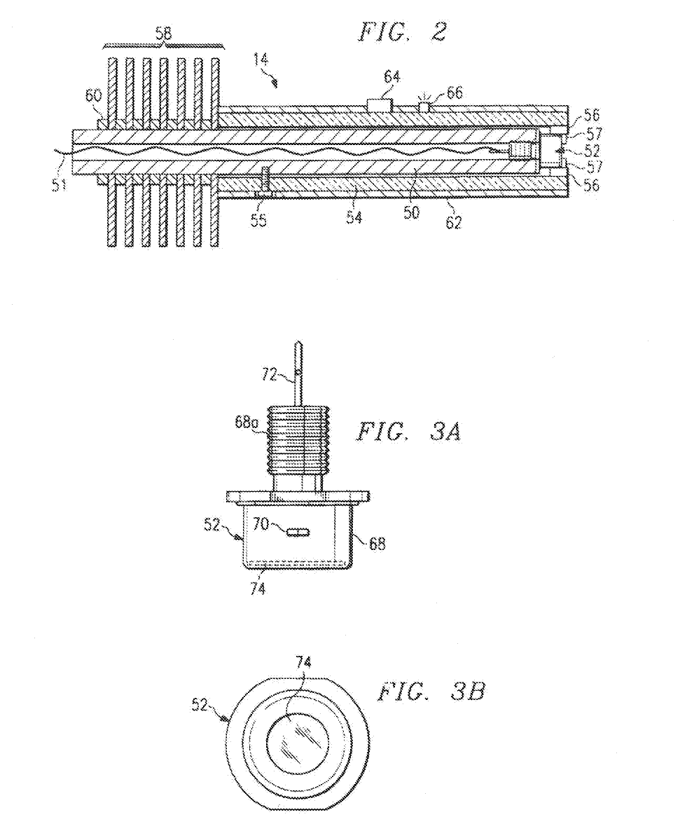

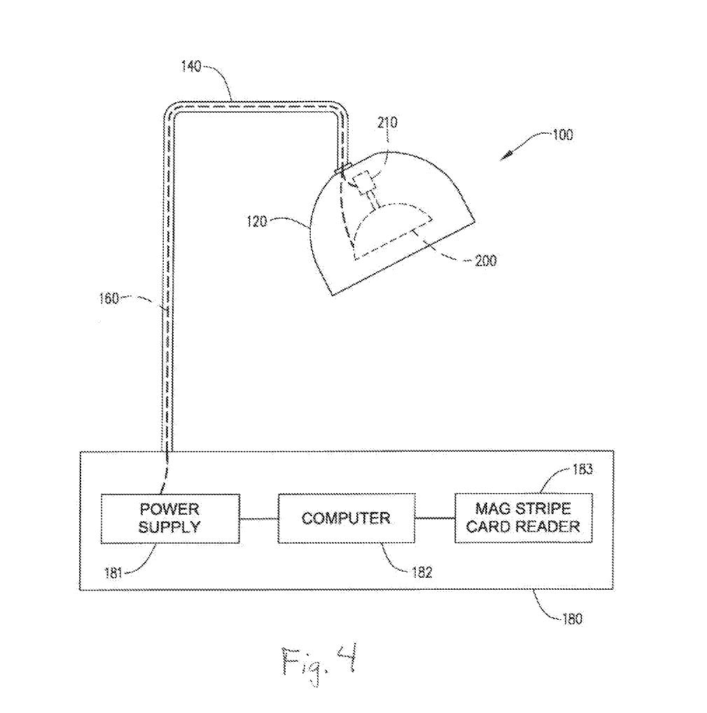

Referring to FIG. 2, the wand 14, sized to be easily manipulated by the user, includes a heat-conductive, metal bar 50. The bar 50 is hollow along its central axis and is threaded on its interior at a first end for receiving a laser resonator 52, described further below with reference to FIGS. 3A and 3B. Wiring 51 extends from the resonator 52 through the hollow axis of the bar 50 for connection to the coaxial cable 16 (FIG. 1). In the preferred embodiment the bar 50 is copper or steel and thus conducts electricity for providing a ground connection for the resonator 52 to the cable 16.

A glass noryl sleeve 54 is placed over the bar 50 for purposes of electrical and thermal insulation. A screw 55 extending through the sleeve 54 anchors the sleeve to the bar 50. As shown, the resonator 52 is recessed slightly within the sleeve 54. An impedance oring 56, formed of a conductive metal, is press-fitted into the end of the sleeve 54 so that when the wand 14 makes contact with tissue, the ring 56 touches the tissue. The ring 56 is electrically connected through the wand 14 to the unit 12. The ring 56 measures impedance by measuring angular DC resistance with an insulator ohmmeter, for example, of the tissue being irradiated by the wand 14 which is then displayed as impedance on the display 42. Any other suitable impedance measurement circuit may be utilized, as will be apparent to one skilled in the art.

A feedback sensor 57 is located in the end of the sleeve 54 for measuring the output of the resonator 52. While not shown, the sensor 57 is connected electronically to the control unit 12 and to a feedback circuit within the control unit. A small percentage of the diode laser light from the resonator 52 is thus detected by the sensor 57 and channeled into the feedback circuit of the control unit 12 to measure and control performance of the resonator. Out-of-specification temperature, power, pulse frequency or duration is thus corrected or the system 10 is automatically turned off.

Multiple metallic fins 58 are placed over the end of the bar 50 and are separated and held in place by spacers 60 press-fitted over the bar 50. The fins 58 act as a heat sink to absorb heat from the laser through the bar 50 and dissipate it into the surrounding air. The spacers 60 placed between each fin 58 enable air to flow between the fins, thereby providing for increased heat transfer from the wand 14.

A casing 62 fits over the sleeve 54 and serves as a hand grip and structure to support a switch 64 and light 66. The switch 64 is used to actuate the wand 14 by the operator wherein the switch must be depressed for the wand to operate. The switch 64 is wired in a suitable manner to the control unit 12 and is used either alone or in conjunction with the foot pedal 22. The light 66 is illuminated when the wand 14 is in operation.

As shown in FIG. 3A, the laser resonator 52 includes a housing 68 having threads 68a configured for matingly engaging the threaded portion of the bar 50 in its first end. An Indium-doped Gallium Arsenide (In:GaAs) semiconductor diode 70 is centrally positioned in the housing 68 facing in a direction outwardly from the housing 68, and is electrically connected for receiving electric current through the threads 68a and an electrode 72 connected to the wiring 51 that extends longitudinally through the hollow interior of the tube 50 (FIG. 2). The amount of Indium with which the Gallium Arsenide is doped in the diode 70 is an amount appropriate so that the diode 70, when electrically activated, generates, in the direction outwardly from the housing 68, low level reactive laser light having, at a power output level of 100-1000 mW, a fundamental wavelength ranging from, depending upon the implementation, about 1000 nanometers (nm) to 10,000 nm in the near-infrared region of the electromagnetic spectrum. Other types of diode semiconductor lasers may also be used to produce the foregoing wavelengths, e.g., Helium Neon, GaAs or the like.

As shown in FIGS. 3A and 3B, a lens 74 is positioned at one end of the housing 68 in the path of the generated laser light for focusing the light onto tissue treatment areas of, for example, 0.5 mm2 to 2 mm2, and to produce in the treatment areas an energy density in the range of from about 0.01 to about 0.15 joules/mm2. The lens 74 may be adjusted to determine depth and area of absorption.

The operating characteristics of the diode 70 are an output power level of 100-1000 mw, a center fundamental wavelength of 1000 nm to 10,000 nm, with a spectral width of about 5 nm, a forward current of about 1500 milliamps, and a forward voltage of about 5 volts at the maximum current.

It is known in a commercially available hair growth stimulation device to provide laser diodes having a wavelength of about 670 nm, activated at an undisclosed wattage. Applicant's prior patents disclose the use of a laser having wavelengths of from about 1,064 nm to about 2,500 nm for medical treatments that do not involve hair growth stimulation. It has been subsequently discovered that laser diodes having a wavelength within the region from about 2500 nm to about 10,000 nm can also be used for the stimulation of hair growth and tissue regeneration, and more specifically wavelengths in the region from about 2500 nm to about 5000 nm, and even more specifically wavelengths of about 3150 nm.

Broadly, the current invention includes systems, devices, and methods for a light source, typically a diode laser, operating in the infrared range at wavelengths of greater than about 2,500 nm and at a low total wattage, preferably less than about 1,000 mw for the total output of the device, and more preferably less than about 500 mw. A laser operating in this range will have a greater dispersion rate than heretofore, thus requiring fewer diodes to cover the same area of scalp stimulation for promoting hair growth. A number of factors govern effective scalp stimulation: laser diode wavelength and power (diode wattage); light beam divergence and dispersion; duration period of laser light application/stimulation; rate of application, i.e. the number of periods per unit of time; and the distance between the diodes and the scalp. While prior art devices provide a substantial space between the diodes and the scalp, the Applicant has found that a minimal spacing may be more effective when using diodes in this infrared range and at low wattage.

For purposes of appetite suppression key acupuncture/acupressure points are located on the ears, face, lower arm (forearm) and hands. The surface of the tissue in the region to be treated is irradiated with the laser beam light to produce the desired therapeutic effect. Because laser light is coherent, a variable energy density of the light of from about 0.01 to 0.15 joules/mm.sup.2 is obtained as the light passes through the lens 74 and converges onto each of the small treatment areas. The energy of the optical radiation is controlled by the power control 30 and applied (for durations such as 1 minute to 3 minutes, continuous wattage or pulsed, for example) as determined by treatment protocols, to cause the amount of optical energy absorbed and converted to heat to be within a range bounded by a minimum absorption rate sufficient to elevate the average temperature of the irradiated tissue to a level which is above the basal body temperature, but which is less than the absorption rate at which tissue is converted into a collagenous substance. The laser beam wavelength, spot or beam size, power dissipation level, and time exposure are thus carefully controlled to produce in the irradiated tissue a noticeable warming effect, which is also limited to avoid damaging the tissue from thermal effects.

The present invention has several advantages. For example, by using an In:GaAs diode laser to generate the laser beam energy, the laser source can be made sufficiently small to fit within the hand-held wand 14, thereby obviating the need for a larger, more expensive laser source and the fiber optic cable necessary to carry the laser energy to the treatment tissue. The In:GaAs diode laser can also produce greater laser energy at a higher power dissipation level than lasers of comparable size. Furthermore, construction of the wand 14 including the fins 58 provides for the dissipation from the wand of the heat generated by the laser source. In addition, while the present example illustrated in FIG. 1 only includes one laser wand 14, it should be understood that multiple laser diodes and wands may be used to treat large patients or to treat multiple acupuncture/acupressure points simultaneously.

It is understood that several variations may be made in the foregoing without departing from the scope of the invention. For example, any number of fins 58 may be utilized as long they dissipate sufficient heat from the wand 14 so that the user may manipulate the wand without getting burned. The setting controls 24 may be used individually or in combination and the information displayed on the displays 26 may vary. Other diode laser structures may be utilized to produce the desired effects.

FIG. 4 depicts another embodiment of the invention 100, which comprises a stationary cap 120 provided for surrounding and covering a patient's head, in a manner resembling a well-known hair dryer. This embodiment of the invention is designed to stimulate acupuncture/acupressure points in the scalp in order to suppress appetite.

The cap 120 may be supported on a cantilevered support 140 to allow the cap 120 to be positioned over and about the head of a patient while maintaining a non-contact spacing between the interior of the cap 120 and the scalp. The patient's head may optionally be supported by an external chair having a neck rest (not shown) so that spacing between the scalp and the interior of the cap 120 may be maintained. The cap 120 may provide stable support for a cap 200 therein, with the cap 200 being actuated for rotation by a motor 210.

A wiring harness 160 may be provided between the cap 120 and a controller 180 that provides control and power to components contained within the cap 120. In the embodiment shown, the wiring harness 160 may be routed through a hollow interior of the cantilevered support 140 for convenience and to protect the wiring harness 160 from snagging or damage. However, the wiring harness 160 may also be attached directly to the cap 120 by means of a coiled cable, a bundle of bound wires, or other means well known to the art.

The controller 180 may include a power supply 181, a computer 182, an optional magnetic stripe card reader 183, and manual controls (not shown). The power supply 181 may be of standard design having sufficient capacity to power a computer 182, actuate the motor 210 within the cap 120 and to drive light sources within the cap 200, as will be described presently. The computer 182 may provide control to the motor and light sources and receive direction from manual controls (not shown) associated with the controller 180. The magnetic stripe card reader 183 may be representative of various input devices well known to the art, which allow data to be provided to and received by the computer 182.

It should be understood that the configuration described above is representative of the inventive device and obvious modifications providing the same functionality may be used within the scope of the invention. For example, in some embodiments, the wiring harness 160 may be replaced by a wireless protocol in which the controller 180 may broadcast control information to a receiving unit located in the cap 120, with the controller 180 and the cap 120 having their own independent power supplies 181. The magnetic stripe card reader 183 may be substituted with a flash memory card or a floppy disk reader. Other obvious modifications may be contemplated as being within the scope of the invention.

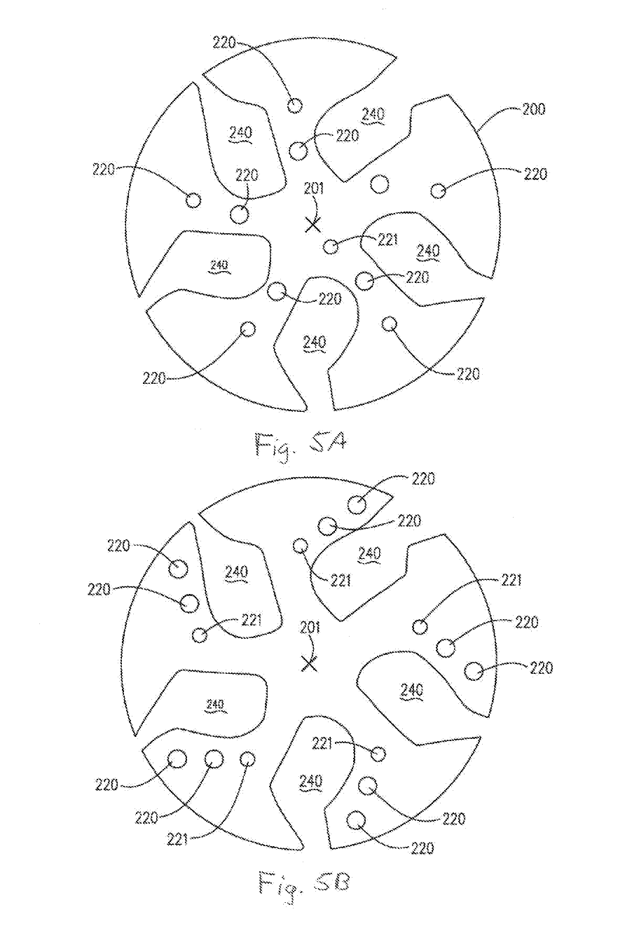

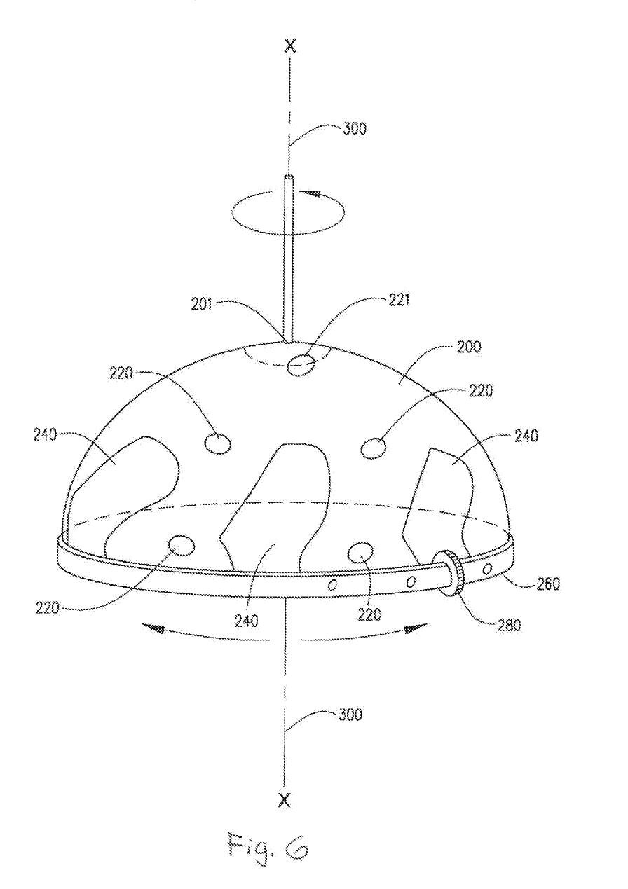

The cap 200 contained within the cap 120 may be of a generally circular aspect. A flattened pattern for the cap 200 is shown in FIGS. 5A and 5B, which has a center of rotation 201. Cutouts 240 may be removed from the flattened pattern to allow the resulting shape to assume a three-dimensional form as by bending or folding the portions remaining between the cutouts 240. The cap 200 may be formed by folding each portion inwardly in the same direction to form what geometrically is known as a spherical cap (FIG. 6), which is defined as the shape resulting from a plane passing through a sphere. The diodes 220 in the cap 200 may be inwardly directed towards the interior of the cap 200. The cap 200 thus formed may be sized to allow its shape to be fitted over and around the patient's head for rotational movement without making firm contact with the patient's head. The spherical cap may extend so far as to form a geometric hemisphere, but preferably the spherical cap forming cap 200 may typically comprise from one-half to one-third of a hemisphere. Cap 200 may be fabricated of a thin, durable flexible material, which can be formed into the spherical cap shape as shown in FIG. 6.

Referring now to FIG. 6, an adjustment strap 260 may be provided about the bottom of cap 200, with a knurled adjustment knob 280 to adjust the shape of cap 200 to accommodate various head sizes, in a well-known manner. In another embodiment, the adjustment strap 260 may be overlapped and secured by using a standard hook-and-loop device that is well known to the industry and sometimes marketed under the trademark Velcro®. Other devices for adjusting and securing the strap to accommodate differing head sizes may be used without departing from the scope of the invention.

Cap 200 may be designed for rotation about an axis 300 that passes through the center of rotation 201. Such rotation may be accomplished through any conventional motor means known to the art. The number of diodes 220, the placement of the diodes 220 about the cap 200, the cyclical sequence of rotational movement, and the actuation of the diodes 220 may be design choices that depend upon the areas of the scalp that are intended to be stimulated for hair growth.

In the embodiment shown in FIGS. 5A, 5B, and 6, five pairs of circumferentially-spaced diodes 220 may be placed so that they flank cutouts 240 in cap 200. An eleventh diode 221 may be located near center of rotation 201. Although only 11 diodes 220, 221 are shown for illustrative purposes, as many as 20 to 30 single diodes 220 may be placed in cap 200 so that they traverse the area of interest on the scalp. Additionally and without departing from the scope of the invention, the site for each diode 220 may comprise a cluster of diodes 220, so that the area traversed by the cluster is broader than the area traversed by a single diode 220. It should also be noted that the spacing of diodes 220, 221, as shown in FIGS. 5A, 5B, and 6, is not to scale and is understood to be for illustration purposes only.

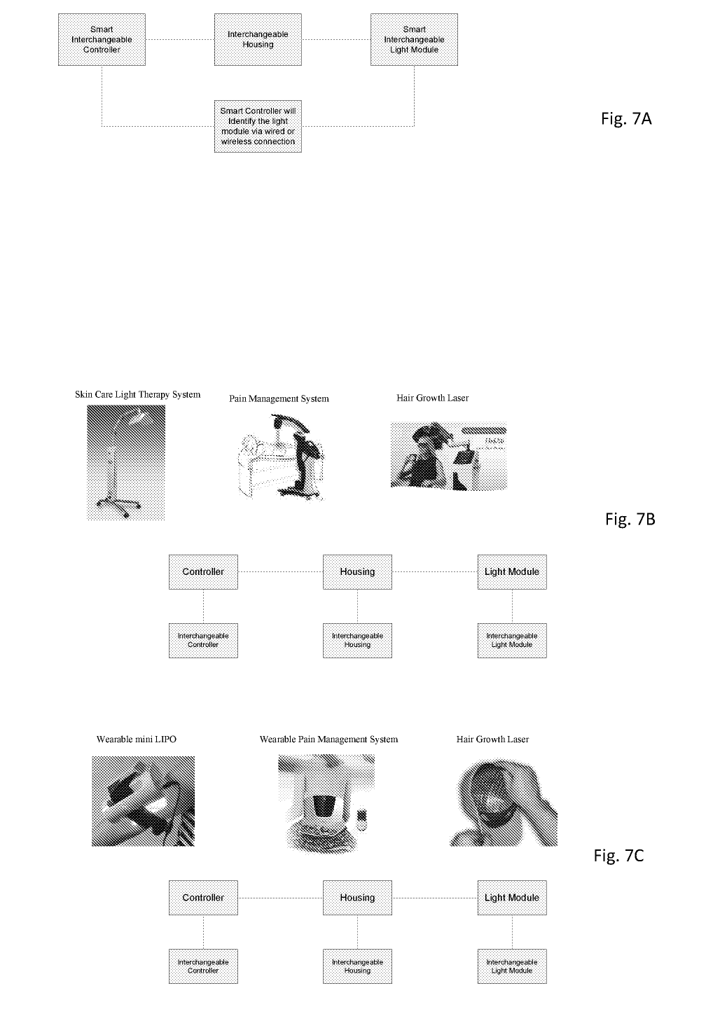

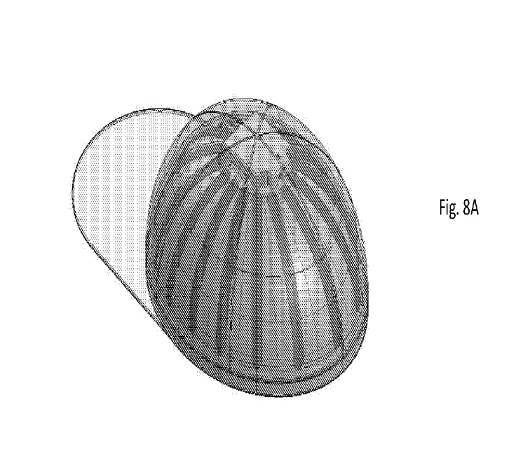

In one embodiment, the invention provides interchangeable elements for application of laser light. This modularity allows greater flexibility in the choice of components used in different therapeutic treatments. FIG. 7 is a diagram of a modular laser system, according to an embodiment of the invention.

Once the smart controller identifies the light module connected to it the smart controller will configure itself for the software to load new control parameters and load the appropriate graphical user interface (GUI). For example, for hair growth laser-a hair laser module might have 48 laser diodes, 72 laser diodes or 96 diodes. When different hair laser module are connected to the smart controller, the controller will re-configure itself so the laser diode power output can be maintain at the same level. This modular reconfiguring can also be applied to different light sources with different wavelengths and power outputs for different therapeutic applications, e.g. hair laser, pain management laser, skin therapy laser, acupressure, etc. When a new light module is connected to the smart controller, the controller will re-configure itself to load specific software with specific user interface and control parameters to control the light module. The model can be applied to both clinical and home devices, as shown in FIGS. 7B and 7C, respectively.

In an embodiment both the clinical and home versions have motor that can rotate the laser bands.
1 The individual laser diode can be selectively turned on/off.
2 The strength of the each laser diode power output can be selectively controlled.
3 With rotatable capability of the earpiece and the helmet/self-standing and with the capability mentioned above, the invention will have the capability to treat a specific area with a programmable period of time and with a programmable laser power for hair growth.
4 Image sensor and blood flow sensor can be added to make the hair growth laser smart
   a The image sensor can be used to scan the scalp then the treatment can focus on the area with less hair density.
   b The blood flow sensor can be used to detect the blood flow in subcutaneous tissue. The blood flow sensor feedback will be used to optimize the treatment time and laser power level.

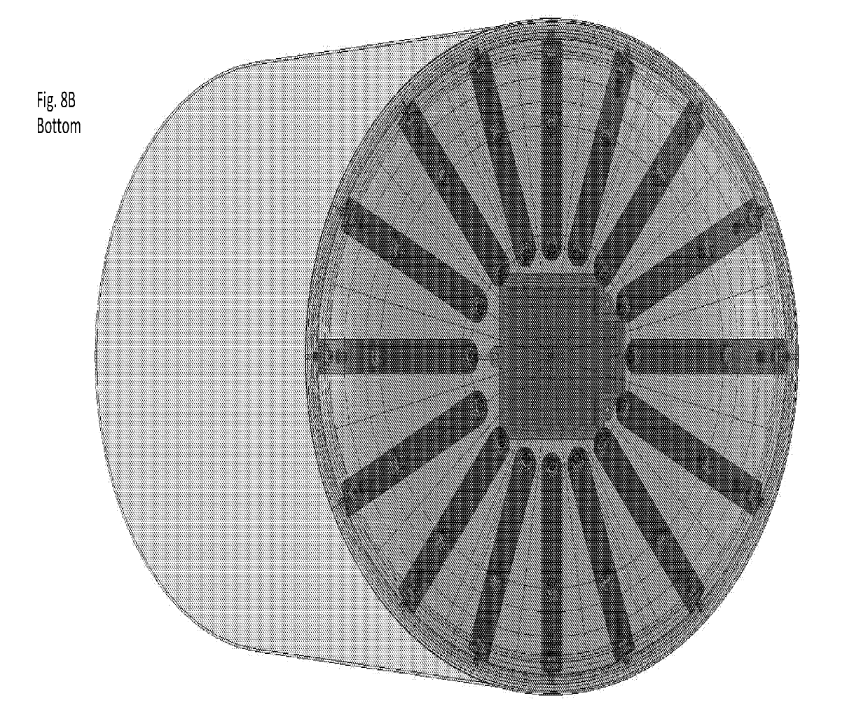

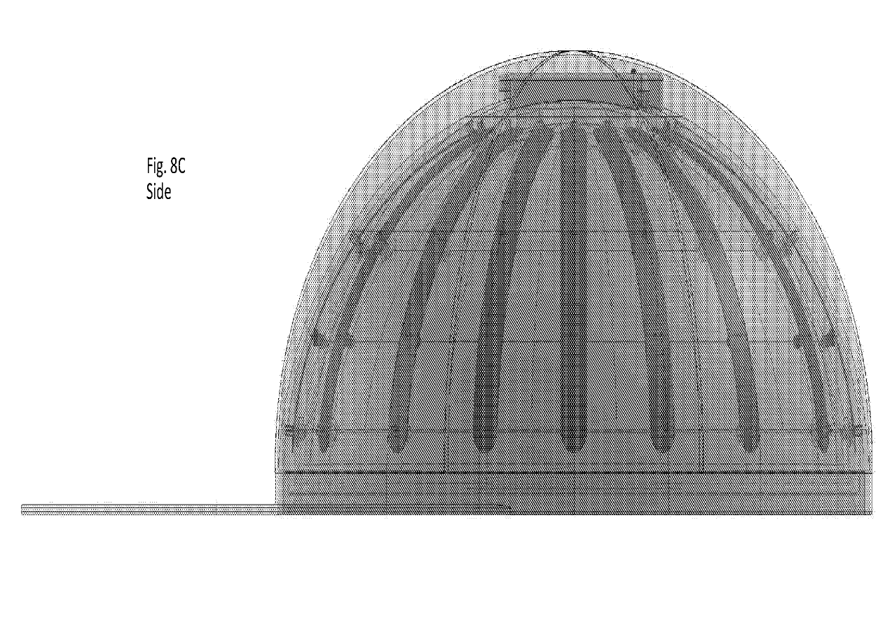

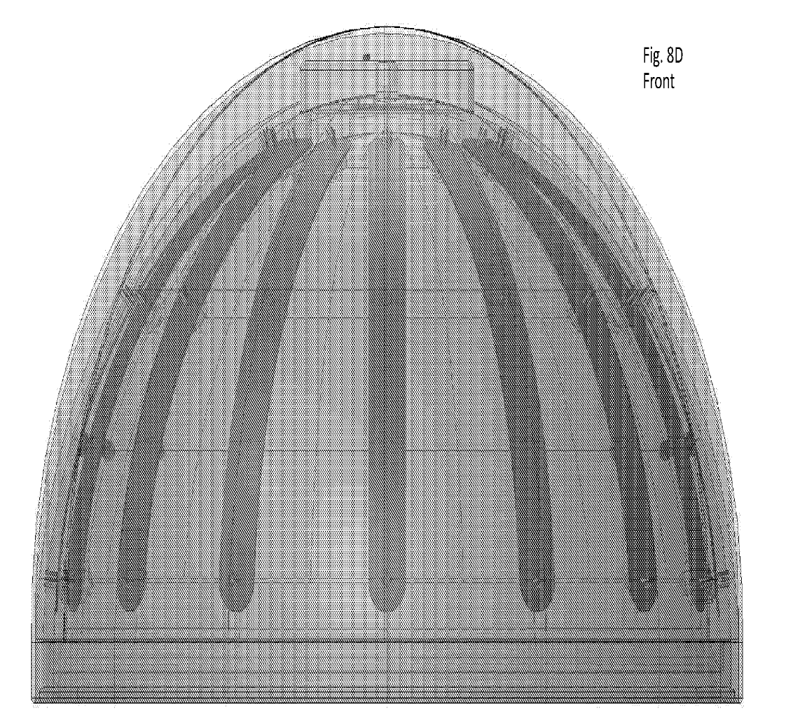

FIGS. 8A-8E show an interchangeable modular laser diode cap in accordance with an embodiment of the invention. The Interchange/Modular cap provides both Clinical and Home version with a modular concept.
1 The Hat is interchangeable:
   a The “hat” can be a soft fabric hat—Home version.
   b The “hat” can be a dome—Clinical version.
2 The frame functions like a snap base plate to allow the laser module to attach to the frame:
   a The frame has built in mechanism to allow the laser module (below) to attach/detach (interchangeable) from the frame.
3 The laser module:
   a Can be a band of lasers—similar to the Helmet Laser Band module
   b Can be a cluster of laser—similar to the cluster of Clinical Laser module
   c Can be a matrix—5×5, 6×6, etc.
   d The laser module will have a smart chip identifier.
   e The laser module is interchangeable.
   f You can MIX the laser module that attach to the frame.
      (i) Different shapes of laser module and different wavelengths and different laser power levels can co-exist in the same system.
4 Sensor Module:
   a Image sensor and blood flow sensor can be added to make the hair growth laser smart
   b The image sensor can be used to scan the scalp then the treatment can focus on the area with less hair density.
   c The blood flow sensor can be used to detect the blood flow in subcutaneous tissue. The blood flow sensor feedback will be used to optimize the treatment time and laser power level.
5 The smart printed circuit board (PCB):
   a The smart PCB can identify the laser module attached to the frame and the PCB.
   b The PCB can auto-configure itself with default values.

The bands inside the cap may contain one or more diodes along its inner surface, each diode being positioned to shine in a direction that is more or less perpendicular to the scalp surface. If two or more diodes are configured, then the distances between adjacent diodes may be equal to each other or the distances between any pair of adjacent diodes may be different from the distance between any other pair of adjacent diodes, without departing from the scope of the invention. The diodes configured within the cap may provide near infrared radiation having a wavelength that is with a region from about 2500 nm to about 10,000 nm, and more preferably within a region from about 2500 nm to about 3500 nm, and even more preferable about 3150 nm. It is also contemplated to utilize 1350+/−20 nm and up to 2500 nm. It is still further understood that greater and less is contemplated.

Each diode may be operated at a power level of from about 0 mw to about 100 mw, with the total power level applied to all diodes on the band being no more than 1000 mw. The power level applied to each diode may be independently controlled without affecting the power level applied to other diodes, without departing from the scope of the invention. Each band within the cap may have a spacing between diodes that differs from the spacing for other bands, in order to provide more complete coverage of the scalp. The moveable bands may be configured with a constant angular displacement from an adjacent moveable band, with all bands moving as a unit.

The light sources of the inventive device described herein for stimulating hair growth may typically be operated at a collective power level of about 500 mw or less. However, there may be certain circumstances where a higher power level is warranted. For example, in the case of cancer patients, the chemotherapy used to treat the cancer will frequently result in hair loss. Such patients have been found to require higher levels of hair follicle stimulation than the normal patient population. These higher levels of stimulation may be provided by power levels that exceed 500 mw for the collective laser light sources but generally not exceeding 1000 mw collectively.

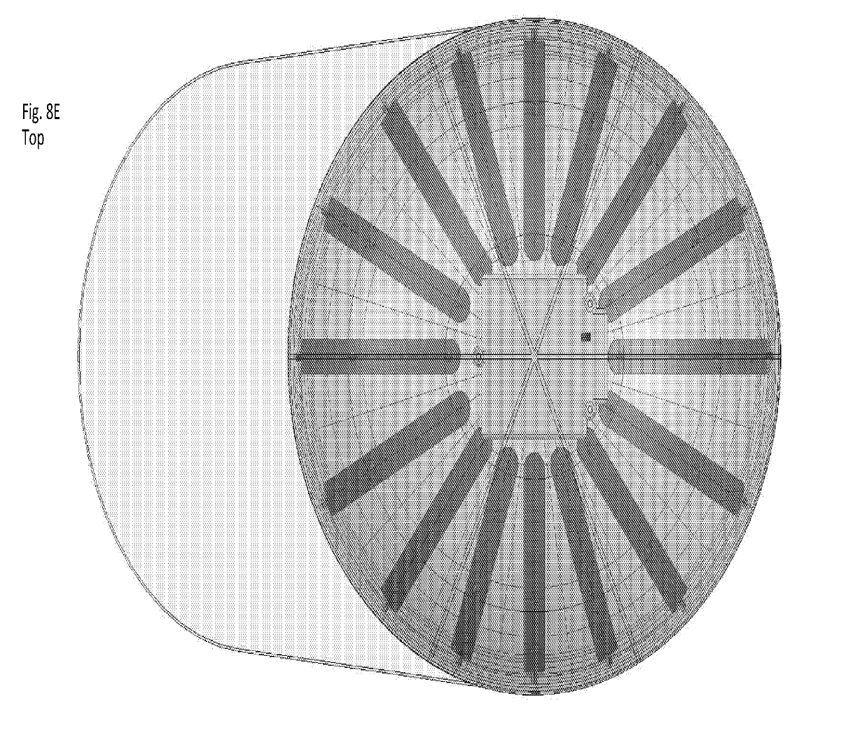

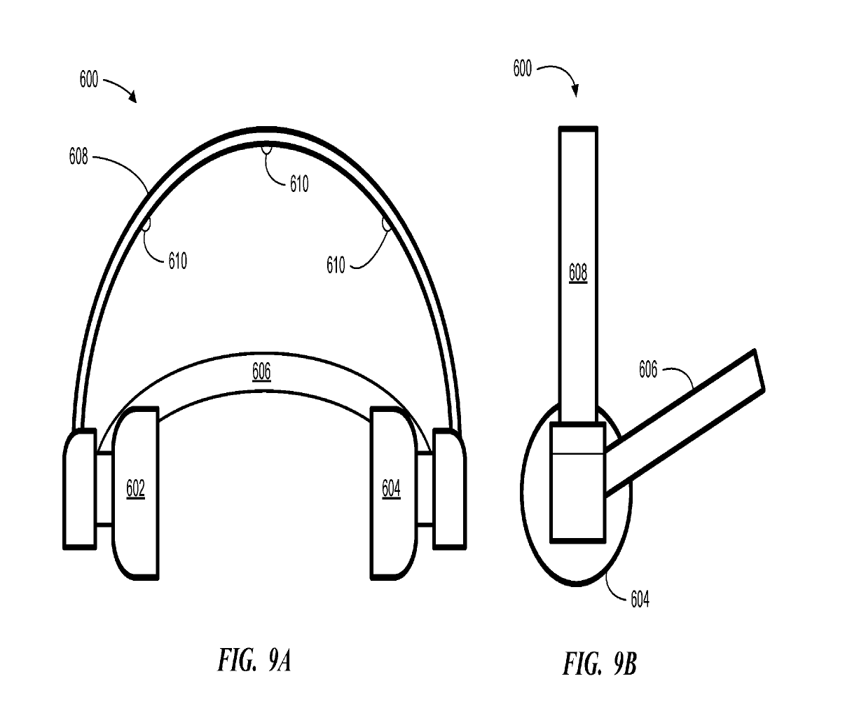

Referring now to FIGS. 9A and 9B, a hair band stimulation device 600 is shown, according to an embodiment of the present disclosure. A pair of ear cups 602, 604 may be fixedly positioned over the ears and maintained at a constant angular displacement about the head by a stabilizer 606. One or both of ear cups 602, 604 may comprise a motor (not shown) that moves a movable band 608 over the scalp in a controlled manner. The moveable band 608 may have one or more light sources 610 along its inner surface for providing radiation to be applied to selected portions of the scalp.

Movable band 608 may be pivotally moved over the surface of the scalp within a certain range. As an example, movable band 608 may rotate over a region of the scalp from about the nape of the neck to about the forehead of the patient. By controlling the extent of travel of movable band 608 over the scalp surface, the power intensity of the diode lasers, and the on/off status of the diode lasers, different areas of the scalp may be targeted for radiation while leaving other areas of the scalp alone.

As shown in FIGS. 9A and 9B, the stabilizer 606 may be a solid band about the back of the head that compressively maintains ear cups 602, 604 over the ears without rotating ear cups 602, 604. The stabilizer 606 may additionally comprise supports (not shown) and other devices that will position ear cups 602, 604 against the shoulder and other body parts. The stabilizer 606 may thus provide a fixed frame of reference within which an angular rotation of the band may take place. The stabilizer 606 shown in FIGS. 9A and 9B may be exemplary and should not be taken as limiting the present disclosure to the embodiment shown in FIGS. 9A and 9B.

Each of ear cups 602, 604 may contain a motor for moving the moveable band 608 over the scalp. In certain embodiments, a single motor may be used on one of ear cups 602, 604 with the other ear cup providing a rotational bearing facilitating angular movement of the moveable band 608, without departing from the inventive concept. Either or both of ear cups 602, 604 may also contain electronic means for providing music, radio, instructions to the patient, and other audio sources to the patient's ears in order to entertain the patient during the radiation process. The ear cups 602, 604 may also have a soft cushion to prevent discomfort during the radiation process.

The moveable band 608 may contain one or more light sources 610 along its inner surface, each light source 610 being positioned to shine in a direction that is more or less perpendicular to the scalp surface. In certain embodiments, the distance between light sources 610 and the patient's scalp may be maintained at all points within a known tolerance range. If two or more light sources 610 are configured, then the distances between adjacent sources 610 may be equal to each other or the distances between any pair of adjacent sources 610 may be different from the distance between any other pair of adjacent sources 610, without departing from the scope of the present disclosure. According to certain embodiments, sources 610 attached to moveable band 608 may provide near infrared radiation having a wavelength that is with a region from about 2,500 nm to about 10,000 nm. Certain embodiments may employ wavelengths within a region from about 2500 nm to about 3500 nm, particularly employing wavelengths near 3150 nm. It is also contemplated to utilize 1350+/−20 nm and up to 2500 nm. It is still further understood that greater and less is contemplated.

Those of skill in the art will understand and appreciate that the above specific frequency ranges are provided only by way of example, and that light sources able to emit light anywhere within the range between approximately 1,330 nm and approximately 10,000 nm may be employed in certain embodiments of the present disclosure. It is possible that frequencies below 1,330 nm may be employed in certain embodiments. It is also possible that frequencies above 10,000 nm may be employed in certain embodiments. Certain embodiments may employ two or more light frequencies, which may be within or outside of the above-referenced frequency ranges.

Each light source may be operated at a power level of from about 0 mw to about 100 mw, with the total power level applied to all diodes on the band being no more than 2700 mw. In certain embodiments, the power level applied to each diode may be independently controlled without affecting the power level applied to other diodes, without departing from the scope of the present disclosure.

Although FIGS. 9A and 9B show a single moveable band 608, multiple bands may be configured for angular movement over the scalp around ear cups 602, 604. Each movable band 608 may preferably have a spacing between light sources 610 that differs from the spacing for other bands 608, in order to provide more complete coverage of the scalp. The moveable bands 608 may be configured with a constant angular displacement from an adjacent moveable band 608, with all bands moving as a unit.

Although the principal embodiment described herein may employ laser diodes as an example light source, there is nothing within the spirit and scope of the present disclosure limiting the light sources to laser diodes, specifically. Depending on the specific application, light may be generated via a variety of laser types, including gas lasers, chemical lasers, dye lasers, metal-vapor lasers, solid-state lasers or semiconductor lasers. It is not necessary that the light used in the present disclosure be generated by a laser. A variety of suitable light sources may be employed in the present disclosure, as will be known to, and appreciated by, those of skill in the art. Further, any suitable devices capable of generating, shifting, refracting, reflecting, polarizing, diverting, focusing or filtering light in such a manner as to provide light at the correct location within the proper frequencies and at the proper level of intensity may be used to generate and direct light in connection with the embodiments disclosed herein. These devices may include, but are not limited to, fiber optics, conduits, mirrors, lenses, prisms and filters.

A controller attached to the device 100 may be adapted to accepted parameters selected by the operator, such as the speed of movement of the band, the angle of rotation, direction (forward or back), actuation of the light sources (i.e., points of time at which a particular light sources may be turned on or off) and power level of each light sources on each band. This set of parameters may be termed a cyclical sequence. The cyclical sequence may be stored in the controller for convenience. A cyclical sequence may be developed for different patterns of hair loss, stored within the controller, and retrieved as needed, depending upon the patient.

In certain embodiments, a periodic cycle may be programmed into the controller that actuates movable band 608 and light sources 610, which will cause movable band 608 to move in a repeated periodic movement over the scalp. The power supplied to each light source may be from about 0 mw to about 15 mw, so that the total power supplied to all light sources 610 does not exceed 500 mw. The movable band 608 may then be allowed to periodically cycle through its programmed course for a fixed length of time. Multiple treatments of this type may be necessary to complete the hair growth stimulation process.

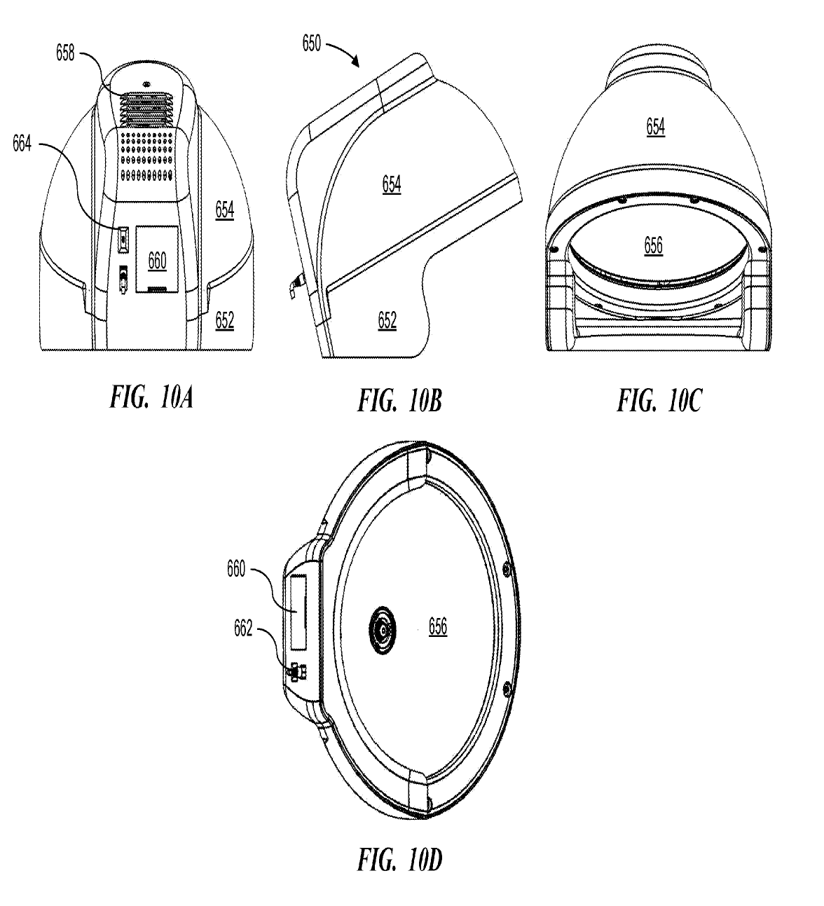

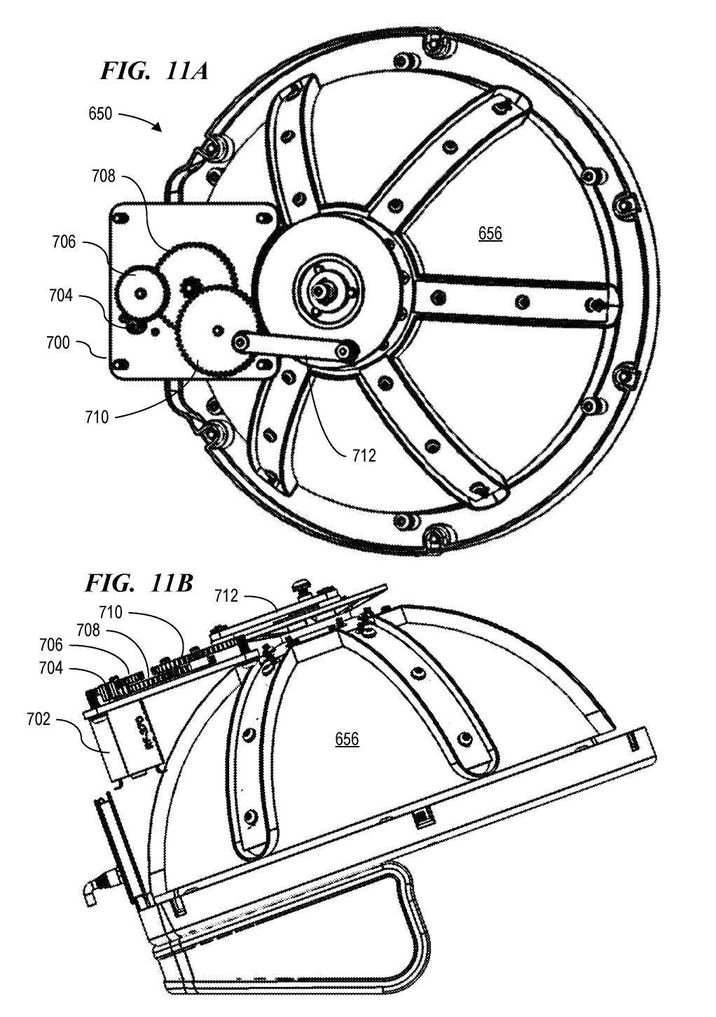

FIGS. 10A-10D show rear, side, front and bottom views, respectively, of a light application helmet according to one embodiment of the present disclosure. Light application helmet 650 comprises a frame 652, an upper housing 654 and an inner dome 656. Frame 652 provides structural support for the various components of light application helmet 650. Upper housing 654 protects the internal components of light application helmet 650, and also provides a measure of safety by preventing objects from becoming entangled in the internal moving parts of helmet 650. Inner dome 656 applies light to the scalp of the patient via a light source or array of light sources (not shown).

Upper housing 654 has a generally-hemispherical shape. The operational mechanisms of helmet 650 are enclosed within the upper anterior portion of upper housing 654. These include both electronic controls and mechanical actuation mechanisms, which are described in further detail below in connection with FIGS. 11A and 11B. The upper anterior portion of upper housing 654 also comprises a vent 658, an access panel 660, a connector 662 and a control switch 664. Vent 658 allows heat to escape from upper housing 654, so as to prevent an overtemperature condition within helmet 650. Access panel 660 provides convenient access to certain operational controls and internal mechanisms without necessitating removal of upper housing 654. Connector 662 provides communication between helmet 650 and an external controller, such as controller 180 described above in connection with FIG. 4. Control switch 664 controls the flow of power to helmet 650.

Inner dome 656 provides therapeutic light to the scalp of a patient via one or more light sources (not shown). The light sources employed may be any of the various types of light sources shown and described in the foregoing disclosure. The light may be applied in a specific pattern, or may be generally diffused evenly across the scalp. Inner dome 656 is rotatably mounted to frame 653 so as to allow inner dome 656 to rotate about a generally-vertical axis of rotation, thereby providing more even distribution of the applied light. In certain embodiments, inner dome 656 may rotate continuously is one direction. In other embodiments, inner dome 656 may oscillate back and forth about its axis of rotation.

Certain embodiments may include additional sensors, which may include image sensors and blood flow sensors to provide closed-loop control of the process. An image sensor may be used to scan the scalp then the treatment can focus on the area with less hair density. A blood flow sensor can be used to detect the blood flow in subcutaneous tissue. Feedback from the blood flow sensor feedback can then be used to optimize the treatment time and laser power level.

In certain embodiments, the unit may include multiple light sources. In certain such embodiments, individual light sources may be selectively turned on or off or the power level varied between light sources.

FIGS. 11A and 11B show a top and side view, respectively, of the light application helmet shown in FIGS. 10A-10D, as it appears with the top cover removed, so as to reveal the rotational mechanism for the inner dome 656.

As seen in FIGS. 11A and 11B, the motion of inner dome 656 is controlled by a motor-driven gear-and-crank mechanism attached to a geartrain frame 700. The mechanism comprises motor 702, secured to frame 700, having a motor pinion 704 connected to its rotor. Motor pinion 704 drives the driven spur gear of first intermediate spur assembly 706, which is also rotatably secured to frame 700. A first intermediate pinion, secured to the bottom of the driven spur gear of the first intermediate spur assembly 706, drives the driven spur gear of second intermediate spur assembly 708, also rotatably secured to frame 700. A second intermediate pinion, secured to the top of the driven spur gear of the second intermediate spur assembly, drives the crank spur gear 710. Crank spur gear 710, secured to the frame 700, is connected to the inner dome 656 by connecting rod 712, thereby allowing a controller, such as controller 180, to control the position of inner dome 656 via motor 702.

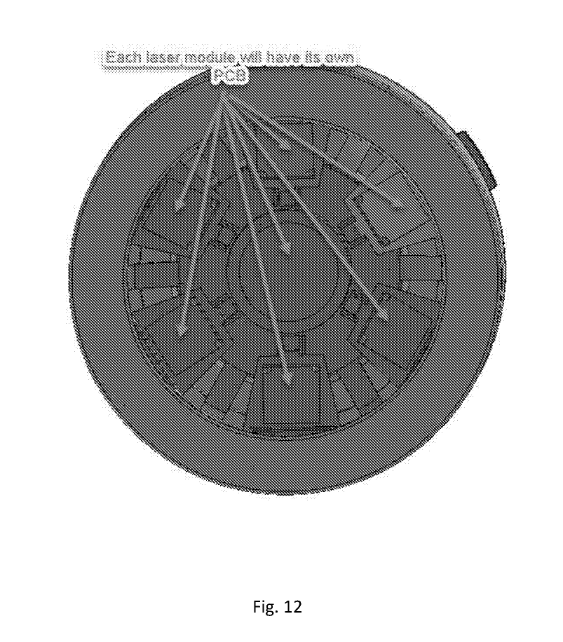

FIG. 12 shows an alternative embodiment in which each laser module has its own printed circuit board (PCB) control unit. This embodiment represents a distributed (modular) design. Instead of one centralized control board to control up to 45 diodes, the embodiment shown in FIG. 12 has seven separate control boards. In one embodiment, six control board are used to control 6 diodes each, and one control board controls nine diodes (6×6+1×9=45 diodes). Each control board will have its own CPU, which means each control board is independent standalone PCB. By changing to the distributed (modular) model, this embodiment minimizes the number of wires to make the clinical much more robust. With this approach, power only need to be brought to the control board.

In another embodiment of the invention, the above apparatuses are operated with laser wavelengths ranging from 800 nm -1350 nm. The power level used for the lasers may be 5 -100 mW. The laser may be either continuous wave or pulsed.

Sensors in the helmet or cap designs described above can determine blood flow to the area targeted for hair growth. An image sensor scans the scalp to determine the area that needs treatment. In this manner, the treatment is focused specifically on the area(s) of the head with less hair density. The blood flow sensor can be used to determine blood flow in subcutaneous tissue to optimize treatment.

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated. It will be understood by one of ordinary skill in the art that numerous variations will be possible to the disclosed embodiments without going outside the scope of the invention as disclosed in the claims.

What is claimed is:

1. An apparatus comprising:
a frame at least partially defining a wearable cap; and
a laser module configured to provide near-infrared radiation.

2. The apparatus of claim 1, wherein the radiation has a wavelength within a region from about 2500 nm to about 10,000 nm.

3. The apparatus of claim 1, wherein the radiation has a wavelength within a region from about 2500 nm to about 3500 nm.

4. The apparatus of claim 1, wherein the radiation has a wavelength within a region from about 2500 nm to about 3150 nm.

5. The apparatus of claim 1, wherein the radiation has a wavelength of 1350+/−20 nm.

6. The apparatus of claim 1,
wherein,
a portion of the laser module is a dome.

7. The apparatus of claim 1,
wherein,
the frame includes a base plate operable to permit the laser module to attach to the frame.

8. The apparatus of claim 1,
wherein,
the frame includes a mechanism operable to permit the laser module to attach and detach from the frame.

9. The apparatus of claim 1, further comprising:
a band of lasers or a cluster of lasers.

10. The apparatus of claim 1, further comprising:
a matrix of lasers including rows and columns in a 5×5 pattern or a 6×6 pattern.

11. The apparatus of claim 1, further comprising:
a smart chip identifier.

12. The apparatus of claim 1, wherein the laser module is interchangeable.

13. The apparatus of claim 1,
wherein,
the laser module includes a plurality of different laser modules, and
the plurality of different laser modules has different shapes, different wavelengths, and/or different laser power levels.

14. The apparatus of claim 1, further comprising:
a sensor module.

15. The apparatus of claim 1, further comprising:
an image sensor and a blood flow sensor.

16. The apparatus of claim 15, wherein the image sensor is operable to scan a scalp of a user to permit a treatment to focus on an area of the scalp with lower hair density than another area of the scalp with greater hair density.

17. The apparatus of claim 15, wherein the blood flow sensor is operable to detect blood flow in subcutaneous tissue, and provide feedback to optimize treatment time and laser power level.

18. The apparatus of claim 1, further comprising:
a smart printed circuit board (PCB),
wherein,
the smart PCB is operable to identify the laser module, and
the smart PCB is operable to auto-configure with default values.
